(19) United States
(12) Patent Application Publication
(10) Pub. No.: US 2024/0156537 A1
(43) Pub. Date: May 16, 2024

(54) HEART COMPRESSION DEVICE AND METHOD

(71) Applicant: Maximum Fidelity Surgical Simulations, LLC, Columbia, MO (US)

(72) Inventor: Joss Fernandez, Columbia, MO (US)

(21) Appl. No.: 18/510,622

(22) Filed: Nov. 15, 2023

Related U.S. Application Data
(60) Provisional application No. 63/425,536, filed on Nov. 15, 2022.

(51) Int. Cl. A61B 34/10; G09B 23/30; G09B 23/32

(52) U.S. Cl. CPC A61B 34/10; G09B 23/303; G09B 23/32; A61B 2034/105

(57) ABSTRACT

A heart compression system and method configured to supplement and/or replace cardiac contractility to mimic in vivo conditions is provided. In an embodiment, the system comprises a sealed primary chamber configured to house a cadaveric heart. Pressure within the primary chamber is selectively adjusted to cause compression of the heart while avoiding direct, mechanical contact. In an embodiment, a heart is placed within a pressurization medium within the primary chamber, and compression of the heart is achieved by selectively increasing and decreasing pressure within the primary chamber. In an embodiment, the system includes a series of sealed entry ports into the primary chamber accommodating insertion of conduits for sealed connection to the vessels and/or chambers of the heart and to a fluid reservoir external of the primary chamber. In an embodiment, the fluid reservoir contains a perfusion fluid for perfusing the heart.

### CROSS-REFERENCE TO RELATED APPLICATIONS

This application claims priority pursuant to 35 U.S.C. 119(e) to U.S. Provisional Patent Application Ser. No. 63/425,536, filed Nov. 15, 2022, the entire disclosure of which is incorporated herein by reference.

### FIELD OF THE INVENTION

The present invention relates generally to systems and methods for medical training, simulation, and study. More specifically, the present invention is concerned with compression of hearts to supplement or replace cardiac contractility for the purposes of medical or surgical simulation or improving cardiac function in a live patient.

### BACKGROUND OF THE INVENTION

Accurate and realistic medical simulations are necessary for effective training and preparation of physicians and other medical personnel. Cadavers and human organs remain important resources for such training because oftentimes, procedures performed on plastic models and/or other animals have significant differences from performing the same procedures on a living, human patient. Consequently, it would be beneficial to have a system for and a method of more closely modeling a living patient or living, human tissue during medical training and research.

While simulations utilizing cadavers can provide for more accurate simulation scenarios, such systems and methods also have their disadvantages. For instance, systems for and methods of creating a surgical model by pumping fluid into a cadaver creates models which are not very mobile. Consequently, such trainings are somewhat limited to “surgical room” training or the like, where cadavers can be prepped and generally do not need to be moved. Unfortunately, not all real-life scenarios are “surgical room” scenarios. Consequently, it would be beneficial to have a mobile model. It would further be beneficial if the mobile model facilitated training for a variety of scenarios, such as “emergency room” scenarios, “accident scene” scenarios, “natural disaster” scenarios, “crime scene” scenarios, “terrorism” scenarios, “battlefield” scenarios, and the like.

Moreover, whole cadavers are expensive and difficult to store, while certain portions of cadaveric tissue tend to be less expensive and easier to store. Many procedures merely require a portion of a cadaver rather than a whole cadaver. Furthermore, it can be expensive and time-consuming to flush cadaveric arterial and/or venous branches and/or to otherwise prepare entire or large portions of a cadaveric circulatory system. Furthermore still, cadaveric circulatory systems can be damaged during preparation, potentially rendering such cadaver unusable with certain methods and/or creating uncertainty or otherwise adversely affecting the feasibility and/or usefulness of a cadaver. Consequently, it would be beneficial to have a system for and method of utilizing portions of a cadaver.

Procedures to repair or replace cardiac valves through catheter-based systems continue to play an increasingly import role in the care of cardiac patients. Nevertheless, cardiac valve replacement device development has primarily utilized live animal studies, despite disparate anatomies from humans, or in the alternative, has utilized artificial valves within synthetic circuit to model heart valve(s). These technologies have their disadvantages. For instance, in addition to the differing anatomy issue, blood of a living animal is opaque, preventing direct visualization during a testing scenario. Also, for synthetic circuit models, the flow of simulated blood fluid does not closely resemble human tissue. As a result, the prior art fails to provide a system for or method of obtaining a direct view of a dynamic heart valve. As a further result, devices either never make it to market or modifications are required after patients have been affected by implantation of the device. Consequently, it would be beneficial to have a system for and methods of obtaining a direct view of heart valves. It would further be beneficial if such methods and procedures included reanimation of a cadaveric heart.

Furthermore, there is a need for a true, functional, whole-heart model to study valve function and compression assist devices. Prior attempts at augmenting or supplementing cardiac contractility have been made, but such prior art required direct mechanical pressure on the heart. This mechanical pressure is an issue because of the friability of heart tissue and the resulting damage it creates. More specifically, the amount of mechanical stress required to simulate the pumping action of a human heart, even at merely 60 beats per minute, rapidly degrades the heart tissue, causing defects that lead to loss of simulated blood fluid. Furthermore, it is very difficult to mimic complete circumferential compression as occurs in the heart, and the time of compression is never perfectly uniform, resulting in distortion of the heart valves. Other prior art systems have utilized pressurization of fluid entering the heart as a method to mimic valve closure. This results in higher ventricular pressures occurring when the ventricle is most distended, which is the opposite of the natural cardiac cycle in which ventricular pressure is highest during compression. Accordingly, this results in distorted valve architecture and poor replication of cardiac function. Consequently, it would be beneficial to have a whole-heart model which accurately demonstrates valve architecture and function without additional mechanical stress on the heart tissue.

Heretofore there has not been available a system or method for heart compression to supplement or replace cardiac contractility with the advantages and features of the present invention.

### SUMMARY OF THE INVENTION

The present invention comprises a heart model system for, and an associated method of, compressing a heart to supplement or replace cardiac contractility. In an exemplary embodiment, the heart model system comprises a heart compression system having primary container defining a primary chamber in which a heart, human or otherwise, is placed. In an exemplary embodiment, the primary chamber is selectively pressurized to cause the heart to contract in a manner which mimics in vivo heart function while avoiding direct contact with the heart tissue by mechanical means.

In an exemplary embodiment, the primary chamber is watertight and configured for housing an amount of incompressible fluid (“Suspension Fluid”) such that the heart is suspended within such Suspension Fluid within the primary chamber. In an exemplary embodiment, the primary container includes and/or is engaged with a selectively movable wall, wherein moving the movable wall inward and outward decreases and increases the volume of the primary chamber, respectively. By deviating the volume of the primary chamber, a pressure within the primary chamber is correspondingly deviated, thereby fluctuating compressive forces acting upon the heart. By fluctuating compressive forces in a rhythmic manner, the system simulates a beating heart. In an exemplary embodiment, the amount, speed, and frequency of movements of the movable wall are all controlled through a programmable logic controller.

In an exemplary embodiment, the heart compression system further comprises a series of sealed, pressure-controlled valves or ports, which allow sealable access into the primary chamber. In an exemplary embodiment, the heart model system further comprises a perfusion system comprising an external fluid reservoir fluidically connected to a series of conduits configured for insertion through the ports of the heart compression system, the conduits being further configured for connection in a sealed manner to chambers and/or vessels of the housed heart, thereby facilitating fluid flow between the fluid reservoir and the heart. In an exemplary embodiment, the external fluid reservoir houses a simulated blood fluid. In some embodiments, the perfusion system includes a pump that is configured for pumping fluid from the reservoir to the heart. In some embodiments, the heart model system is configured such that compression of the heart results in pumping simulated blood fluid back to the reservoir via the connected conduits.

In some embodiments, the present invention provides a system and method for closely modeling a living patient and/or living human tissue during medical training and research.

In some embodiments, the present invention comprises a mobile heart model. In embodiments, the present invention accommodates simulations in emergency room scenarios, accident scene scenarios, natural disaster scenarios, crime scene scenarios, terrorism scenarios, battlefield scenarios, and the like, among other simulation scenarios.

In some embodiments, the present invention comprises systems and methods of utilizing a heart portion of a cadaver for medical simulation.

In some embodiments, the present invention comprises a system and method providing direct views of heart valves. In embodiments, the system and method of the present invention utilizes a reanimated cadaveric heart. In embodiments, the present system and method provides direct views of heart valves of a reanimated cadaveric heart.

In embodiments, the present invention comprises a whole-heart model which accurately demonstrates valve architecture and function without unnecessary mechanical stress on the utilized heart tissue.

The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention and various features thereof.

### BRIEF DESCRIPTION OF THE DRAWINGS

Embodiments of the present invention are described in detail below with reference to the attached drawing figures, wherein:

FIG. 1 shows a top, plan view of a heart compression system embodying the present invention, the heart compression system shown in a pressurized configuration with a heart submerged within a pressurization medium within a primary chamber and a movable wall portion in an expanded configuration.

FIG. 2 shows a top, plan view of the heart compression system of FIG. 1, the heart compression system shown in depressurized configuration with the movable wall portion in a retracted configuration.

FIG. 3 shows an upper, perspective view of an embodiment of the heart model system of the present invention, the heart model system shown with a perfusion system being in fluidic communication with the heart compression system of FIG. 1.

FIG. 4 shows an upper, perspective view of the heart compression system of FIG. 1, a top wall of a primary container of the heart compression system being removed to provide better visibility of the primary chamber.

FIG. 5 shows a top, plan view of the heart compression system of FIG. 1 in the configuration of FIG. 4.

FIG. 6 shows an upper, perspective view of a fluid reservoir and fluid inflow and outflow conduits of the perfusion system of an embodiment of the heart model system.

The drawing figures do not limit the present invention to the specific embodiments disclosed and described herein. The drawings are not necessarily to scale, emphasis instead being placed upon illustrating the principles of the invention.

### DETAILED DESCRIPTION OF THE PREFERRED EMBODIMENTS

As required, a detailed embodiment of the present invention is disclosed herein; however, it is to be understood that the disclosed embodiments are merely exemplary of the principles of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

The present invention comprises a heart model system and method configured to supplement and/or replace cardiac contractility to mimic in vivo conditions. In an exemplary embodiment, the heart model system includes a heart compression system having a sealable primary container that defines a sealable primary chamber for receiving and housing a heart, whether human or otherwise. In an exemplary embodiment, the primary chamber houses a whole heart. In alternative embodiments, the heart compression system houses portions of a heart. In embodiments, the heart compression system utilizes all or a portion of a cadaveric heart. In other embodiments, the heart compression system utilizes all or a portion of a heart of another animal. In embodiments, the heart compression system utilizes an anatomically correct heart, including the left atrium, right atrium, left ventricle, right ventricle, and corresponding vessels and valves of an anatomic heart. In further embodiments, the system utilizes a synthetic whole heart or synthetic heart portion.

In an exemplary embodiment, the system further includes a series of sealed entry ports in communication with the primary chamber, entry ports being configured to accommodate access to the heart, such as for a medical procedure and/or to facilitate perfusion of the heart, such as by using a perfusion system of the present invention. In some embodiments, the entry ports facilitate sealed insertion of a series of conduits into the chamber, the conduits being configured for sealed attachment to the chambers and/or vessels of the heart and to a fluid reservoir external of the chamber. In an exemplary embodiment of the present invention, the fluid reservoir contains a volume of perfusion fluid, such as simulated blood fluid, for perfusion through the housed heart during contraction of the heart caused by the present system. In some embodiments, the perfusion fluid includes water, real blood, plasma, dimethyl sulfoxide (DMSO), albumin, glycerol, artificial blood, starch, combinations thereof, or any other fluid now known or later discovered or developed to simulate blood. In exemplary embodiments, the simulated blood fluid comprises Simblood, Envivoflush, Envivolyte, or Envivoblood, each of the foregoing a product sold by Maximum Fidelity Surgical Simulations, LLC or its affiliates, or combinations thereof. In an exemplary embodiment, the viscosity of the perfusion fluid can be adjusted to better mimic blood.

In an exemplary embodiment, the primary chamber is substantially a rectangular prism shape, with opposed top and bottom walls and four side walls (i.e., a front wall, back wall, left wall, and right wall) extending therebetween. In other embodiments, the chamber is substantially a cube shape, a sphere shape, a spheroid shape, a cylinder shape, an oval prism, a triangular prism, or any other geometric shape configured to house a heart.

In an exemplary embodiment, the primary container is sealed and pressurized using a pressurization system of the present invention. In an exemplary embodiment, the pressure within the primary chamber is selectively adjusted to cause compression of the heart housed within the chamber to mimic in vivo heart function while avoiding mechanical means of direct contact to compress the heart. In an exemplary embodiment, the heart is suspended within a pressurization medium within the chamber. In embodiments, the pressurization medium comprises an incompressible gas, liquid, or semisolid. In some embodiments, the pressurization medium and the perfusion simulated blood fluid comprise the same fluid. In other embodiments, the pressurization medium and the perfusion simulated blood fluid are different. In some embodiments, the pressurization medium fluid includes a heart preservation solution.

In some embodiments, the primary container includes an adjustable wall, the adjustability of which is controlled by the pressurization system. In some embodiments, the adjustable wall is a movable wall that is moveable between retracted and extended configurations, such as by utilizing a screw drive with a motor, by fluctuating pressure within a secondary chamber opposed to the primary chamber, or by otherwise changing the forces acting upon the moveable wall so as to encourage movement of the same. In other embodiments, the adjustable wall includes a moveable wall portion rather than the entire wall being moveable. In some embodiments, the pressurization system includes a bladder and a compressor for controlling the configuration of the adjustable wall, with expansion and contraction of the bladder resulting in movement of the adjustable wall between extended and retracted configurations, respectively. In some such embodiments, the volume of the pressurization medium within the primary chamber remains constant or substantially constant throughout oscillation of the adjustable wall between its extended and retracted configurations, thereby generating oscillating compressive forces on the heart so as to mimic a beating heart.

In some embodiments, the primary chamber is in fluid communication with a fluid reservoir for holding compression fluid. In some such embodiments, a fluid conduit extends between the fluid reservoir and the primary chamber, thereby facilitating flow of compression fluid into and out of the primary chamber such that pressure in the primary chamber increases and decreases, respectively. In some embodiments, at least a portion of the pressurization medium includes or is interchangeable with the compression fluid, thereby facilitating increasing and decreasing (effectively or actually) the volume of the pressurization medium within the primary chamber. In this way, pressure within the primary chamber is increased and decreased, thereby increasing and decreasing compressive forces on the heart. In some such embodiments, the volume of the primary chamber remains constant or substantially constant throughout oscillation of the fluid in and out of the primary chamber, thereby generating oscillating compressive forces on the heart so as to mimic a beating heart.

In some embodiments, the pressurization system includes a secondary container positioned within the primary chamber, the secondary container being configured to expand and contract, thereby decreasing and increasing net volume of the primary chamber. In some such embodiments, the volume of the pressurization medium within the primary chamber remains constant or substantially constant throughout expansion and contraction of the secondary container, thereby generating oscillating compressive forces on the heart so as to mimic a beating heart.

In an exemplary embodiment, the present invention further comprises a processor electrically connected to the pressurization system and a programmable logic controller for control and adjustment of pressure within the primary chamber and the amount, speed, and frequency of compression. In an exemplary embodiment, the pressurization system further includes sensors for detecting pressure, temperature, and/or fluid volume within the primary chamber.

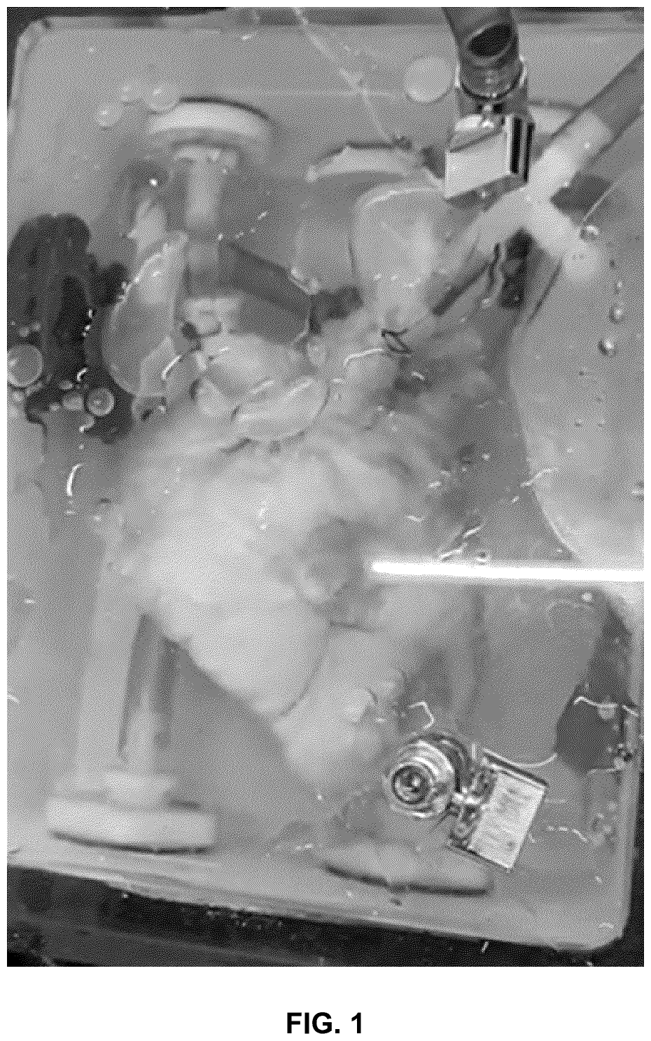

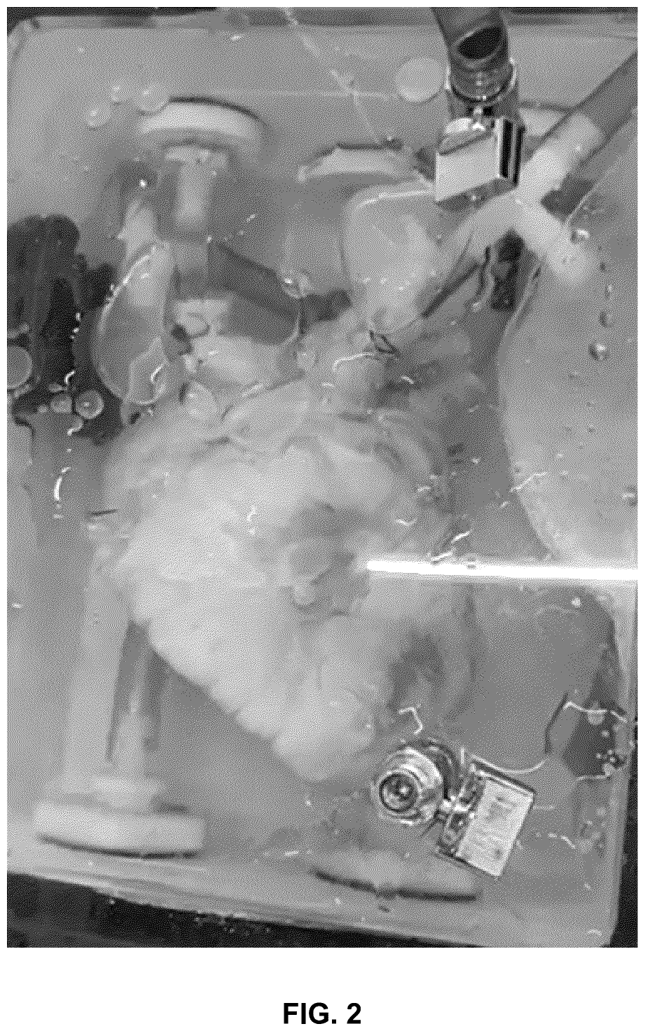

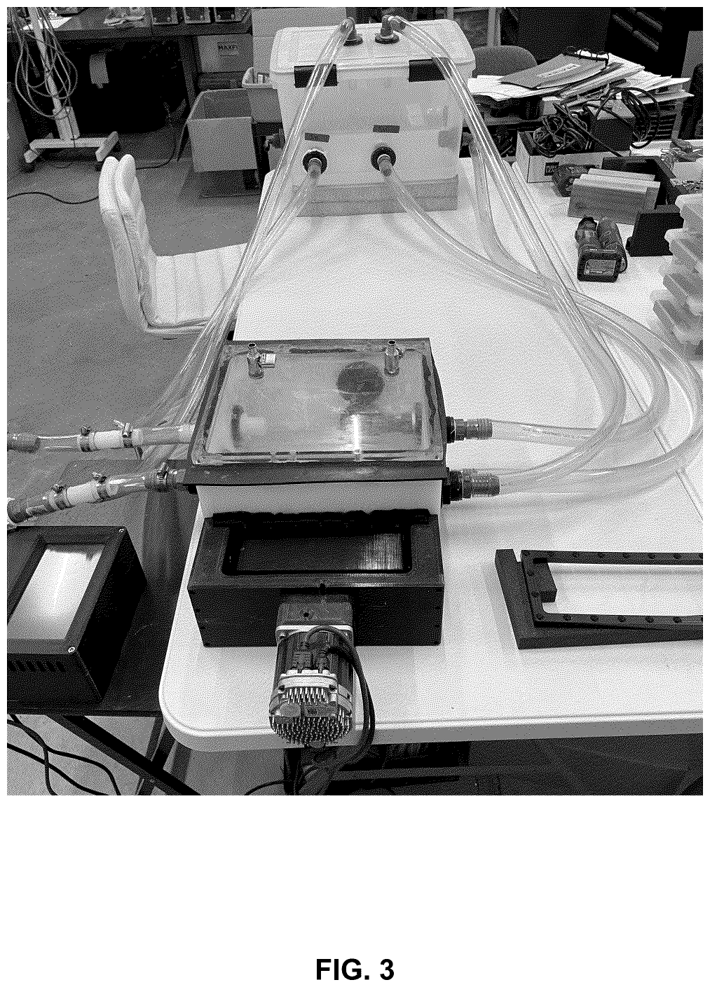

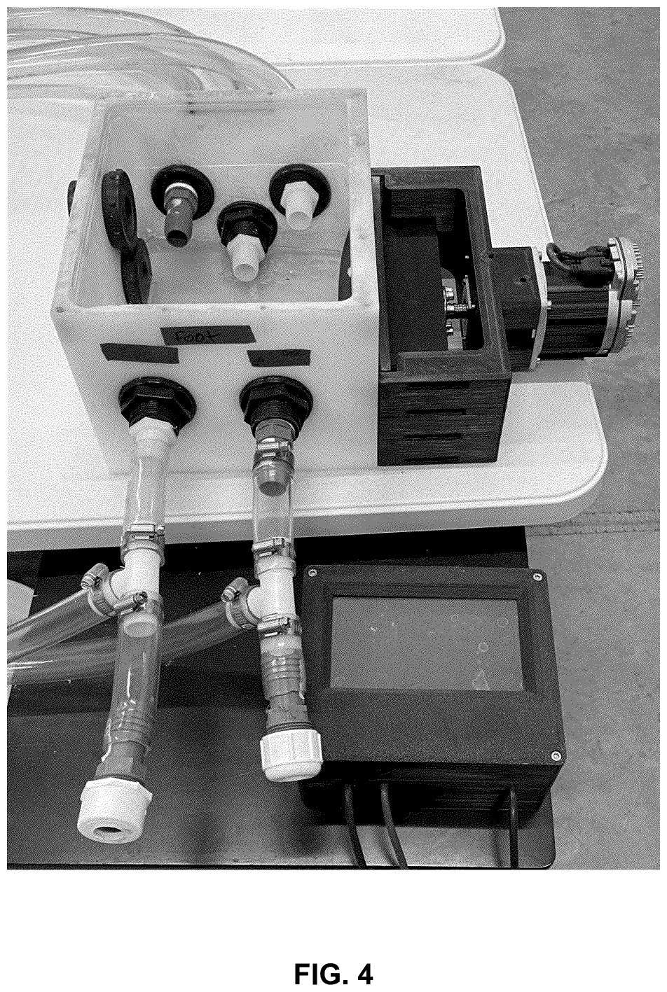

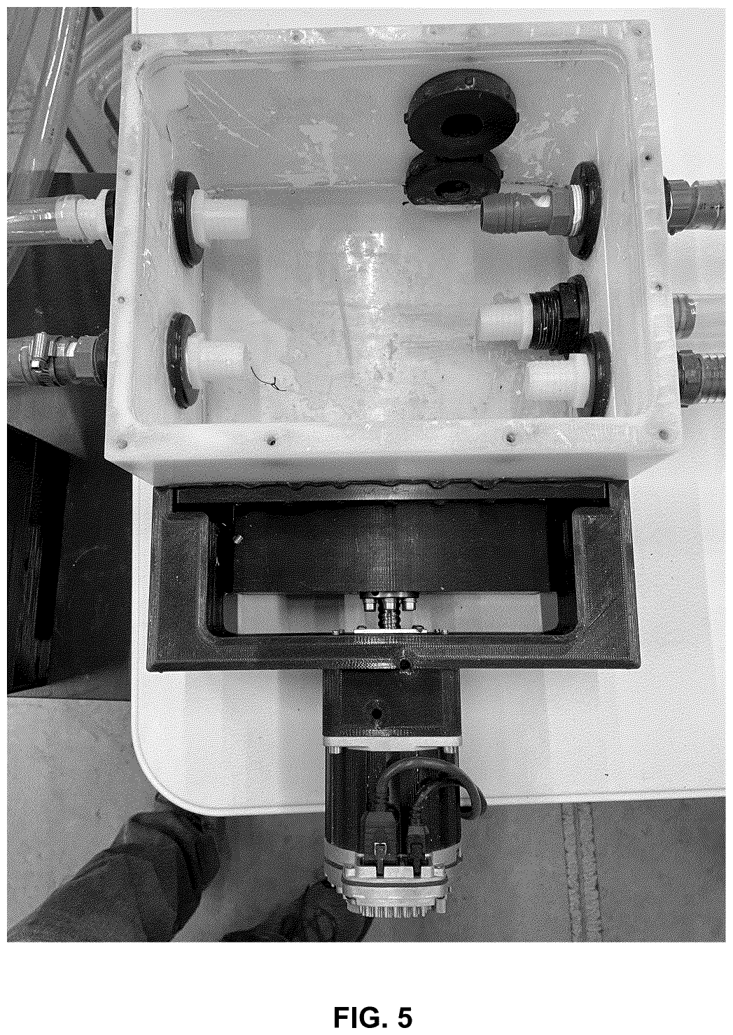

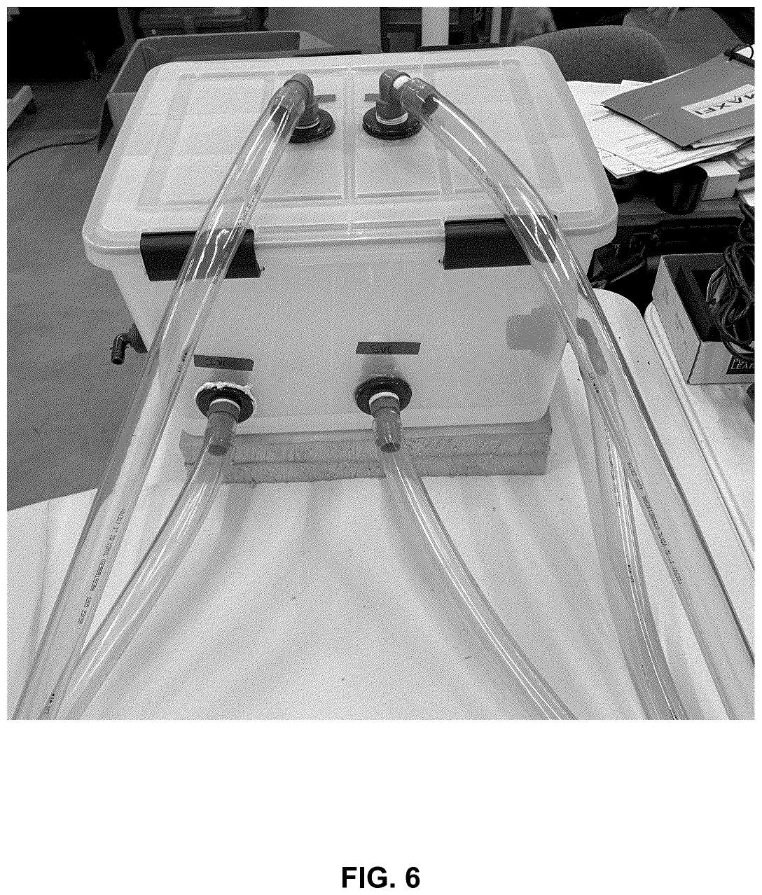

FIGS. 1-6 show an exemplary embodiment of a heart compression system of the present invention, with a whole heart positioned within a primary chamber having substantially a rectangular prism shape. In this exemplary embodiment, the primary chamber of the present invention is configured to be hermetically sealed around a heart. The system further comprises a series of sealed ports to allow access to the heart within the chamber, as needed for live heart and medical simulation scenarios.

In this embodiment, the heart is placed within a pressurization medium within the chamber, and compression of the heart is achieved by selectively alternating between increased and decreased pressure within the chamber around the heart. In this embodiment, such pressure differential is achieved by the chamber having at least one movable wall which is selectively expandable into the chamber. In such embodiment, the heart is submerged and sealed in an incompressible fluid, and the fluid surrounding the heart is displaced and therefore pressurized with a fixed volume of the chamber by expanding the movable wall into the chamber and thereby compressing the chamber. Then the movable wall is retracted, thereby lowering the pressure within the chamber. In an exemplary embodiment, the movable wall is alternatingly expanded and retracted, which causes the heart to compress and contract in a manner which simulates in vivo conditions while avoiding direct mechanical contact with the fragile outer heart tissue.

In an embodiment, the movable portion of the movable wall comprises an inflatable bladder which selectively inflates and deflates with air as controlled by a connected motor. In other embodiments, the wall is configured to physically slide inward and outward with respect to the chamber interior, such sliding wall mechanism connected to a motor. In an embodiment, the sliding wall mechanism comprises a screw linear actuator assembly, a belt driven actuator assembly, or any other linear actuator. In another embodiment, the pressurization fluid submerging the heart is displaced by a pressurized gas bladder positioned within the closed chamber. In another embodiment, the chamber includes air fluid around the heart, and a liquid, gas, or other fluid is used to pressurize the air around the heart.

In an exemplary embodiment, the pressure within the chamber is approximately between 100 and 200 psi when system is in a high compression configuration, such as when the movable wall is expanded into the chamber (i.e., during compression), and is approximately between 0 and −50 psi when the system is in a low compression configuration, such as when the movable wall is retracted (i.e., during expansion of the chamber).

In an exemplary embodiment, system oscillates between high and low compression configurations at a selected rate, such as between 30 and 120 times per minute, thereby mimicking a selected heart rate. In an exemplary embodiment, expansion of the movable wall displaces between 10 and 200 mL of fluid. In an exemplary embodiment, the movable wall moves at a speed of approximately one centimeter in between 100 and 500 milliseconds. In an exemplary embodiment, the movable wall is connected to a motor which is connected and controlled by a programmable logic controller. In an exemplary embodiment, the speed, frequency, and amount of movement in relation to the chamber interior can be adjusted using a touch screen or toggle buttons on the controller. In embodiments, the controller is connected to a power source, such as a battery, an alternating current source, or a direct current source.

In an exemplary embodiment, the movable wall comprises a selectively inflatable bladder connected to an air pump which includes a motor and is connected to the programmable logic controller. In an exemplary embodiment, the inflatable bladder is made of neoprene. In other embodiments, the inflatable bladder is made of silicone, natural rubber, latex, or any other similar synthetic material now known or later developed. In some embodiments, the chamber includes a thin, second layer of material between the inflatable bladder and chamber interior to further prevent leaks from the bladder into the chamber interior.

In an exemplary embodiment, the chamber is made of a radiolucent material to allow for x-rays of the heart to be performed during testing of devices. In some embodiments, at least a portion of the chamber is transparent to allow for visualization of the heart valves.

In an exemplary embodiment, a top wall is placed on the chamber and sealed to the side walls of the chamber. In exemplary embodiments, the chamber is equipped with a deairing valve and/or a valve to allow for the influx and draining of pressurization fluid to surround the heart.

In an exemplary embodiment, a graphical user interface (GUI) allows for the manipulation of a pulse generator of the motor controller. In an embodiment, the heart rate is controlled by cycles of the motor per minute, which is controlled by the logic controller. Preferably, the depth of the stroke and/or stroke volume are also controlled and adjustable. In an exemplary embodiment, a pressure sensor and/or gauge are fitted to outflow channels of the chamber and displayed as a form of feedback. In further embodiments, pressure feedback data is utilized by the system processor to create an intelligent circuit where the independent variables are blood pressure and heart rate.

In some embodiments, the heart model system includes a perfusion system for perfusing the heart model. In some such embodiments, the perfusion system includes a fluid reservoir for holding perfusion fluid and a plurality of fluid conduits extending from the fluid reservoir and through sealable access port associated with the primary chamber. At least some of the fluid conduits are configured to engage with the heart such that they are in fluid communication with internal cavities of the heart. In some embodiments, the fluid perfusion system is configured to bias perfusion fluid towards the heart, such as by way of a pump, hydraulic head, or other means of biasing fluid now known or later developed. In some such embodiments, the biasing force is such that flow of the perfusion fluid oscillates between flowing towards or away from the heart as compression on the heart oscillates from a low level to a high level, respectively.

In an exemplary embodiment of the present invention, airtight access ports of the sealed chamber are provided for access into the chamber and to the heart. In embodiments, the chamber access ports are sealed by one-way valves. In embodiments, these one-way valves may be electronically actuated by the controller or passive. In some embodiments, such valves prevent the backflow of fluid from the heart, such as but not limited to through the veins during compression of the heart. In an exemplary embodiment, conduits are inserted into the chamber through such ports and connected in sealing relation to chambers or vessels of the heart. Such conduits are connected at their other ends to a fluid reservoir external to the chamber which houses a simulated blood fluid. In an exemplary embodiment the fluid reservoir and conduits are configured for perfusing simulated blood fluid through the heart within the chamber. In some embodiments, the fluid reservoir includes an attached pump, and in other embodiments, there is no attached pump. In further embodiments, one or more endoscopes are inserted into the chamber through one or more sealing access ports and inserted into a portion of the heart in sealing relation.

In an exemplary embodiment, the conduit connections from the reservoir into the chamber are pressurized to increase the flow rate to the heart using a pump. The conduits to the chamber from the reservoir may also comprise valves to reduce back flow or they may be without such valves. In embodiments, the size of the conduits will vary depending on which veins or arteries of the heart are being filled. The size of the conduits may range from ⅛ inch to 3 inches in diameter to match sizes of heart vessels. The flow rate for a given pressure is determined by the Bernoulli's equation. In the present invention, flow rate can be controlled by the electronic controller or alternatively, can be controlled in its simplest form by adjusting the height of the fluid reservoir relative to the chamber.

To mimic flow into the heart it is further important to control the pressure within the veins and the flow rates. For consideration, the typical venous pressure in a human is between 5 mmHg and 15 mmHg, and the typical flow rate of blood is between 4 and 6 L/min. During a cycle of the heart rhythm, the heart chambers need to be filled within diastole. At a heart rate of 120 beats per minute, this gives only 250 milliseconds to fill 120 mL within the right and left sides of the heart at a pressure of only 10 mmHg. Accordingly, the volume at low pressure necessitates large bore access to the heart. In embodiments of the present invention, several methods are deployed to create large bore access to the heart. In embodiments, polyester tube grafts are sewn using polypropylene sutures, such as but not limited to Prolene sutures, to the heart as conduits with large bores. In embodiments, such tube grafts range in size from 8 mm to 36 mm depending on the heart size and the particular artery or vein. In further embodiments, plastic tubes are anchored to the artery(ies) or vein(s) of the heart. Here, the tubes are inserted into the vessels and a suture tie is used to cinch the vessel from the outside around the tube. In further yet embodiments, a combination of plastic tubes with polyester grafts on the inside are used to secure conduits to the heart. In additional embodiments, various adhesives and pastes can be used to seal conduits to the vessels, directly to the chamber, or to a heart chamber.

Compression and/or pressurization of the chamber causes pressurization of all contents of the chamber. Arteries and veins, which have different diameters than the ventricles of the heart, will be compressed with the same force. As described by Laplace's law, the tension in the walls of these smaller diameter vessels is much lower than the larger diameter ventricle. Without remediation of this issue, the result is collapse of vessel walls. Accordingly, the present invention utilizes different features to address this issue in different embodiments. In some embodiments, the interior of one or more vessel is supported by a tube inserted which is made of plastic(s) or other material(s). In such embodiments, the tube may have solid walls or consist of corkscrew spirals. In further embodiments, the exterior of one or more vessel is supported by a tube made of plastic(s) or other material(s). Such tube may have solid walls or consists of corkscrew spirals or individual rings sewn to the vessel. In further yet embodiments, one or more vessel is plasticized to harden the walls and sustain more compression. In additional embodiments, one or more vessel is encased in a resin or acrylic to protect against compression.

In an exemplary embodiment, displacement of fluid within the chamber from movement of the movable wall causes pressurization of the heart, which causes the heart mitral and tricuspid valves to close and the aortic and pulmonic valves to open. The flow of fluid from a fluid reservoir that is elevated between 5 and 20 centimeters above the level of the chamber causes filling of the heart during diastole (i.e., when the heart is not compressed within the chamber). This filling of the heart causes the mitral and tricuspid valves to open. Back pressure from fluid flowing out of the heart to the elevated reservoir causes closure of the aortic and pulmonic valves.

In exemplary embodiments, access is provided to the superior vena cava and inferior vena cava of the heart. In embodiments, the superior vena cava access is used to perfuse the right atrium from a reservoir from which pressure and/or height determine the venous pressure in the right side of the heart. In embodiments, the inferior vena cava access is available for instrumenting the heart valves with test devices. In embodiments, the inferior vena cava access is kept hemostatic by way of an access valve. In embodiments, access is provided to the pulmonary artery for outflow of the right ventricle. In embodiments, access for inflow to the heart is provided by a cannulation of the left atrial appendage.

In alternative embodiments, access for inflow to the heart is provided by ventricular cannulation. In embodiments, access to the carotid and/or subclavian arteries provides an opportunity to mimic diastolic pressure (i.e., back pressure). Such mimicked diastolic pressure is accomplished by utilizing a pressured reservoir. In embodiments, descending aortic access is used for instrumentation of the heart. In embodiments, an access is also provided for the introduction of a transesophageal probe behind the heart. In further embodiments, two other additional accesses are used to place intracardiac cameras to image the heart from the inside.

In exemplary embodiments, the system of the present invention comprises one or more reservoirs outside of but fluidically connected to the chamber. In embodiments, fluid from one or more reservoirs is delivered into the chamber through hermetically sealed bulk heads through the wall of the chamber. In an exemplary embodiment, each reservoir comprises a fluid receptacle and a hot water bath to regulate the temperature of the perfusion fluid, as further described in U.S. Patent Application Publication No. 2020/0365057, the entirety of which is hereby incorporated by reference. In embodiments, the reservoir(s) further comprises a return tube to the top of the reservoir with a downspout into the fluid in the reservoir, preventing air from entering the return tube.

In exemplary embodiments, access to the system chamber with medical device(s) is provided through valved conduits. In embodiments, these valve conduits have quadra-fold valves to retain any fluid. Such valve conduits allow instruments to pass through the valve without leaking around them because of the flexibility of the valves around the instrument as it penetrates the valve.

In exemplary embodiments, endoscopic cameras are placed through valve conduits into the heart from outside the chamber to allow for direct visualization of the inside of the heart within the present system.

In exemplary embodiments, pressure sensors are placed inline with fluid tubes to allow transduction of pressures within the system. In embodiments, these pressures are recorded and used as feed into the computer processor. In embodiments, an algorithm is used to determine the optimal stroke rate, stroke speed, and stroke volume for the required pressure. In embodiments, machine learning is used to calculate the compliance of the heart under different loads. This in turn can be used to map the changes in aortic and pulmonary artery pressure in both systole and diastole with given dependent parameters of stroke speed, stroke volume, and stroke rate.

In embodiments of the present invention, the system is utilized in augmenting poorly functioning cardiac tissue in live patients. In embodiments, a heart is healed within a relatively un-distensible pericardial sac. In embodiments, pressurizing the fluid around the heart in a synchronized fashion with the cardiac cycle augments contractility. This improved cardiac function can help patients bridge or altogether avoid cardiac transplantation.

Certain terminology will be used in the description for convenience in reference only and will not be limiting. For example, up, down, front, back, right, and left refer to the invention as orientated in the view being referred to. The words “inwardly” and “outwardly” refer to directions toward and away from, respectively, the geometric center of the aspect being described and designated parts thereof. Forwardly and rearwardly are generally in reference to the direction of travel, if appropriate. Additionally, anatomical terms are given their usual meanings. For example, proximal means closer to the trunk of the body, and distal means further from the trunk of the body. Said terminology will include the words specifically mentioned, derivatives thereof, and words of similar meaning.

As used in this specification and the appended claims, the singular forms “a”, “an”, and “the” include plural references unless the context clearly dictates otherwise. Thus, for example, a reference to “a method” includes one or more methods, elements, and/or steps of the type described herein and/or which will become apparent to those persons skilled in the art upon reading this disclosure and so forth.

As used in this specification and the appended claims, the use of the term “about” means a range of values including and within 15% above and below the named value, except for nominal temperature. For example, the phrase “about 3 mM” means within 15% of 3 mM, or 2.55-3.45, inclusive. Likewise, the phrase “about 3 millimeters (mm)” means 2.55 mm-3.45 mm, inclusive. When temperature is used to denote change, the term “about” means a range of values including and within 15% above and below the named value. For example, “about 5° C.,” when used to denote a change such as in “a thermal resolution of better than 5° C. across 3 mm,” means within 15% of 5° C., or 4.25° C.-5.75° C. When referring to nominal temperature, such as “about −50° C. to about +50° C.,” the term “about” means ±5° C. Thus, for example, the phrase “about 37° C.” means 32° C.-42° C.

Unless defined otherwise, all technical and scientific terms used herein have the same meaning as commonly understood by one of ordinary skill in the art to which this invention belongs. Although any systems, elements, methods and materials similar or equivalent to those described herein can be used in the practice or testing of the present invention, the preferred systems, elements, and methods and materials are now described. All publications mentioned herein are incorporated herein by reference to describe in their entirety.

“Substantially” means to be more-or-less conforming to the particular dimension, range, shape, concept, or other aspect modified by the term, such that a feature or component need not conform exactly. For example, a “substantially cylindrical” object means that the object resembles a cylinder but may have one or more deviations from a true cylinder. “Comprising,” “including,” and “having” (and conjugations thereof) are used interchangeably to mean including but not necessarily limited to, and are open-ended terms not intended to exclude additional, unrecited elements or method steps.

Changes may be made in the above methods, devices and structures without departing from the scope hereof Many different arrangements of the various components depicted, as well as components not shown, are possible without departing from the spirit and scope of the present invention. Embodiments of the present invention have been described with the intent to be illustrative and exemplary of the invention, rather than restrictive or limiting of the scope thereof. Alternative embodiments will become apparent to those skilled in the art that do not depart from its scope. Specific structural and functional details disclosed herein are not to be interpreted as limiting, but merely as a representative basis for teaching one of skill in the art to employ the present invention in any appropriately detailed structure. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described.

It is also to be understood that the following claims are intended to cover all of the generic and specific features of the invention herein described, and all statements of the scope of the invention which, as a matter of language, might be said to fall therebetween.

Having thus described the invention, what is claimed as new and desired to be secured by Letters Patent is:

1. A heart compression system for compressing a heart, the heart compression system comprising:
a primary container defining a primary chamber that is configured to receive and house the heart and a pressurization medium; and
a pressurization system for deviating pressure within the primary chamber, thereby deviating compressive forces acting on the heart.

2. The heart compression system of claim 1, wherein the primary container comprises an adjustable wall, the adjustable wall being a moveable wall or comprising a moveable wall portion, wherein the adjustable wall is moveable between an expanded configuration and a retracted configuration, thereby decreasing and increasing the volume of the primary chamber, respectively, and wherein the pressurization system is configured to oscillate the adjustable wall between its expanded and retracted configurations.

3. The heart compression system of claim 2, wherein:
said pressurization system comprises an inflatable bladder in fluid communication with an air compressor;
said expanded configuration of the adjustable wall comprises said inflatable bladder being in an inflated configuration; and
said retracted configuration of the adjustable wall comprises said inflatable bladder being in a deflated configuration.

4. The heart compression system of claim 3, wherein:
said motor is electrically connected to a programmable logic controller configured for controlling said motor and thereby movement of said movable wall between said expanded configuration and said retracted configuration.

5. The heart compression system of claim 4, wherein:
said programmable logic controller is configured to control speed, frequency, and inflation volume of said inflatable bladder.

6. The heart compression system of claim 4, further comprising:
a user interface connected to said programmable logic controller and configured to allow adjustment of pressure within said primary chamber.

7. The heart compression system of claim 1, wherein:
a top wall of said primary container is transparent to allow for visualization into said primary chamber.

8. The heart compression system of claim 7, wherein:
a bottom wall and at least one side wall of said primary container is transparent to allow for visualization into said chamber, wherein the bottom wall is displaced vertically from the top wall and the at least one side wall extends vertically between the top wall and the bottom wall.

9. The heart compression system of claim 1, further comprising the pressurization medium, wherein:
said pressurization medium comprises an incompressible fluid.

10. The heart compression system of claim 9, wherein:
said incompressible fluid is a clear liquid to facilitate visualization of the heart.

11. The heart compression system of claim 1, further comprising a sealable access port extending through a wall of the primary container, thereby facilitating fluid communication between the heart positioned within the primary chamber and a perfusion system positioned outside of the primary chamber

12. The heart compression system of claim 11, further comprising the perfusion system, wherein the perfusion system comprises:
a fluid reservoir for holding perfusion fluid; and
a fluid conduit extending from said fluid reservoir and through said sealable access port,
wherein the fluid conduit is in fluid communication with internal cavities of the heart when the perfusion system is in an engaged configuration,
wherein the fluid perfusion system is configured to bias perfusion fluid towards the heart when compression on the heart is at a low level, and
wherein the fluid perfusion system is configured to receive perfusion fluid from the heart when compression on the heart is at a high level.

13. The heart compression system of claim 12, wherein the biasing force for biasing perfusion fluid towards the heart is controlled by controlling the head of the profusion fluid within the fluid reservoir.

14. The heart compression system of claim 12, wherein the biasing force for biasing perfusion fluid towards the heart is controlled by controlling a pump in fluid communication with the profusion fluid within the fluid reservoir.

15. The heart compression system of claim 1, wherein said pressurization system comprises:
a fluid reservoir for holding compression fluid; and
a fluid conduit in fluid communication with the fluid reservoir and the primary chamber, thereby facilitating flow of compression fluid in and out of the primary chamber such that pressure in the primary chamber increases and decreases, respectively,
wherein the pressurization medium comprises compression fluid.

16. The heart compression system of claim 1, wherein said pressurization system comprises a secondary container positioned within the primary chamber, the secondary container being configured to expand and contract, thereby decreasing and increasing net volume of the primary chamber, respectively, and wherein decreasing and increasing net volume of the primary chamber increases and decreases pressure within the primary chamber, respectively.

17. The heart compression system of claim 1, wherein the primary container is radiolucent so as to facilitate x-rays of the heart while using the heart compression system for a procedure, wherein the procedure is at least one of testing a device, training personnel, and observing representative heart function.

18. The heart compression system of claim 1, further comprising an enforcement devices for supporting a blood vessel of the heart, thereby preventing collapse of the blood vessel when compressive forces acting on the heart are at a high level, wherein the blood vessel is at least one of the aorta and the vena cava, and wherein enforcement device comprises at least one of a tube inserted into the blood vessel, a hardened wall of the blood vessel, and an enforcing material encasing the blood vessel.

19. A method of compressing a heart, the method comprising:
positioning the heart within a primary chamber of a heart compression system;
filling the primary chamber with a pressurization medium such that the heart is immersed within the pressurization medium; and
oscillating pressure within the primary chamber, thereby deviating compressive forces acting on the heart.

20. The method of claim 19, further comprising:
performing a medical simulation or medical device test utilizing the heart within said primary chamber.

21. The method of claim 19, further comprising:
connecting the heart to a perfusion system;
biasing perfusion fluid towards the heart when compression on the heart is at a low level; and
receiving perfusion fluid from the heart when compression on the heart is at a high level.

22. The method of claim 21, wherein the biasing force for biasing perfusion fluid towards the heart is controlled by controlling the head of perfusion fluid within a fluid reservoir.

23. The method of claim 19, further comprising x-raying the heart while using the heart compression system for a procedure, wherein the procedure is at least one of testing a device, training personnel, and observing representative heart function, and wherein the primary container is radiolucent so as to facilitate x-raying the heart.

24. The method of claim 19, further comprising enforcing a blood vessel of the heart, thereby preventing collapse of the blood vessel when compressive forces acting on the heart are at a high level, wherein the blood vessel is at least one of the aorta and the vena cava, and wherein enforcing the blood vessel comprises at least one of inserting a tube into the blood vessel, hardening a wall of the blood vessel, and encasing the blood vessel with an enforcing material.
